(19) United States
(12) Patent Application Publication
(10) Pub. No.: US 2024/0424248 A1
(43) Pub. Date: Dec. 26, 2024

(54) LABIAL RETENTION DEVICE FOR USE IN URINARY CATHETER PLACEMENT

(71) Applicants: Tanara Boursiquot, Brookhaven, PA (US); Kristen Karlovich, Pittsburgh, PA (US)

(72) Inventors: Tanara Boursiquot, Brookhaven, PA (US); Kristen Karlovich, Pittsburgh, PA (US)

(21) Appl. No.: 18/747,570

(22) Filed: Jun. 19, 2024

Related U.S. Application Data
(60) Provisional application No. 63/634,222, filed on Apr. 15, 2024.
(60) Provisional application No. 63/522,014, filed on Jun. 20, 2023.

(51) Int. Cl. A61M 25/00

(52) U.S. Cl. CPC A61M 25/0017

(57) ABSTRACT

A labial retention device includes a body defining a pair of wings defining therebetween a central opening permitting visualization of urethral and vaginal openings when the device is in contact with a genital region of female anatomy. The body's rear surface bears skin-adhering adhesive. The body may define sidewalls for retaining labia and/or other bodily tissue in displaced positions, and may include an anterior tab and/or a posterior tab to aid in securement and/or act as a shield to prevent unwanted bodily contact. The wings of the body may be joined to form a closed loop encircling the central opening, or be joined such that the central opening is not encircled by the wings. During use, labial/urogenital tissues are displaced and adhered to the rear surface of the labial retention device, which in turn retains the displaced labia/tissues in a spread/displaced position to aid in visualization and catheter placement purposes.

### CROSS-REFERENCE TO RELATED APPLICATIONS

This application claims the benefit of priority, under 35 U.S.C. § 119(e), of U.S. Provisional Patent Application No. 63/522,014, filed Jun. 20, 2023, and of U.S. Provisional Patent Application No. 63/634,222, filed Apr. 15, 2024, the entire disclosures of which are hereby incorporated herein by reference.

### FIELD OF THE INVENTION

The present invention relates generally to a labial retention device that can be used to displace and retain labia of female genitalia, and that can be used in urinary catheterization procedures to facilitate catheter placement with ease and improved accuracy.

### DISCUSSION OF RELATED ART

Urinary catheterization is often required in both clinical and home health care settings. Various catheters (e.g., a Foley or straight catheter) and catheter insertion techniques are well-known in art. Catheterization is typically ordered by a treating physician for patients who retain urine or require alternative measures to void the urinary bladder without contaminating or saturating the skin, as would be the case for someone with a rectal or buttock wound. Catheterization is largely a nursing process, meaning nurses are predominantly the clinicians who insert the catheters.

Proper urinary catheterization of females can be particularly challenging. A typical female catheterization process requires the nurse to use an aseptic or sterile technique, with one hand spread the bilateral labia, differentiate the vagina from the urethra, with labia manually spread open by one of the nurse's hands, and with the other hand insert the catheter into the urethra without breaking the sterile technique.

Such urinary catheterization is subject to a catheter-associated urinary tract infection risk, which may be even higher for females that are of advanced age, have experienced long term chronic catheter use, and/or are diabetic. Additionally, the female labia and surrounding tissues represent a dark moist environment ripe with bacteria. Further, females have labia that are of different sizes, and at the time of catheterization, some may be swollen and even rigid depending on the clinical picture of the individual. Further, the female urethra and vagina are not uniformly situated across females, which can pose challenges to identifying them properly for catheter insertion purposes. In many instances, the introduction of rectal bacteria can contaminate the urethra, the catheter itself, and otherwise increase the risk of infection.

There are additional challenges apart from the female anatomy that further complicate successful catheter insertion. Patients who suffer from obesity, arthritis, immobility, and weakness challenge the nurse's ability to insert the catheter without complication. In the home care setting, nurses are confronted with multiple challenging factors: a patient may sleep in a recliner chair and seek to be catharized in situ, which is not ideal for access or visualization during the catheterization procedure. Further still, poor lighting, lack of available assistance from another person, or the patient's laying on a regular queen or king-size bed without mobility features can also prevent catheterization insertion challenges. All of the above-mentioned factors have a real and potential risk of causing the nurse to violate the sterile technique, thereby increasing the risk of urinary tract infection.

Further still, the provision of catheter supplies is insurance driven. Some insurance companies allow only the number of supplies necessary for the intervention. In such a case, if a doctor ordered a catheter to be changed once per month, the insurance company may approve use of only one catheter per month. By way of example, during catheterization, a nurse may struggle to spread the labia, may inaccurately visualize/identify the urethra, and while attempting to insert the catheter using the “sterile technique,” may erroneously insert the catheter into the vagina. With no other supplies in the home health care setting, it is likely that the nurse will remove the now bacteria-laced catheter from the vagina and reinsert it into the urethra. In this case, although the catheterization task is complete, bacteria has been introduced into the urethra that will result in a urinary tract infection.

What is needed is a device that can be used to assist a clinician in performing a urinary catheterization process on females, and in maintaining a sterile technique during the catheterization process.

### SUMMARY

The present invention provides a labial retention device that can assist in performing a urinary catheterization process on females, and in maintaining sterile technique during the catheterization process. More particularly, the labial retention device can be used to retain displaced labia during the urinary catheterization process, to assist in visualizing and differentiating aspects of the female anatomy to aid in proper identification of the urethra for catheterization placement purposes, and to free a person to use both hands to hold and manipulate the catheter itself during the catheterization process to ensure a successful aseptic insertion.

A labial retention device in accordance with the present invention includes a body defining a pair of wings joined at least at a first junction. The wings define therebetween a central opening sized and shaped to permit visualization of the urethra and vaginal openings of typical human female anatomy through the central opening when the device is in contact with a genital region of female anatomy. The body/wings have a front surface and a rear surface, and a suitable skin-adhering adhesive is supported on said the surface of the body. The body may define sidewalls extending generally transversely to the front surface and away from the rear surface of the body. The body may include an anterior tab and/or a posterior tab. The wings of the body may be joined to form a close loop encircling the central opening, or be joined such that the central opening is not encircled by the wings. During use, labial/urogenital tissues are displaced and adhered to the rear surface of the labial retention device, which in turn retains the displaced labia/tissues in a spread/displaced position to aid in visualization and catheter placement purposes.

### BRIEF DESCRIPTION OF THE FIGURES

An understanding of the following description will be facilitated by reference to the attached drawings, in which:

FIG. 1 is a front view of a labial retention device in accordance with an exemplary embodiment of the present invention;

FIG. 2 is a right side view of the labial retention device of FIG. 1;

FIG. 3 is a right side view of an alternative labial retention device in accordance with an alternative embodiment of the present invention;

FIG. 4 is a front view of another alternative labial retention device in accordance with another alternative embodiment of the present invention;

FIG. 5 is a right side view of the labial retention device of FIG. 4;

FIG. 6 is a rear perspective view of the labial retention device of FIG. 4;

FIG. 7 is a front view of the labial retention device of FIG. 4, shown in use; and

FIG. 8 is a top view of an exemplary catheterization kit including a labial retention device and suitable for use in performing a urinary catheterization procedure, in accordance with the present invention.

### DETAILED DESCRIPTION

The present invention provides a labial retention device 100 that can be used to assist a clinician in performing a urinary catheterization process on females, and in maintaining sterile technique during the catheterization process. More particularly, the labial retention device can be used to retract and retain the labia during the urinary catheterization process, to assist the clinician in visualizing the female anatomy (e.g., both the vagina and the urethra), and to assist the clinician in differentiating the urethra from the vagina to proper identify the urethra for catheterization purposes, and to eliminate the need for the clinician to use one hand to spread the labia and thereby to free the clinician to use both hands to hold and manipulate the catheter itself during the catheterization process to ensure a successful aseptic insertion.

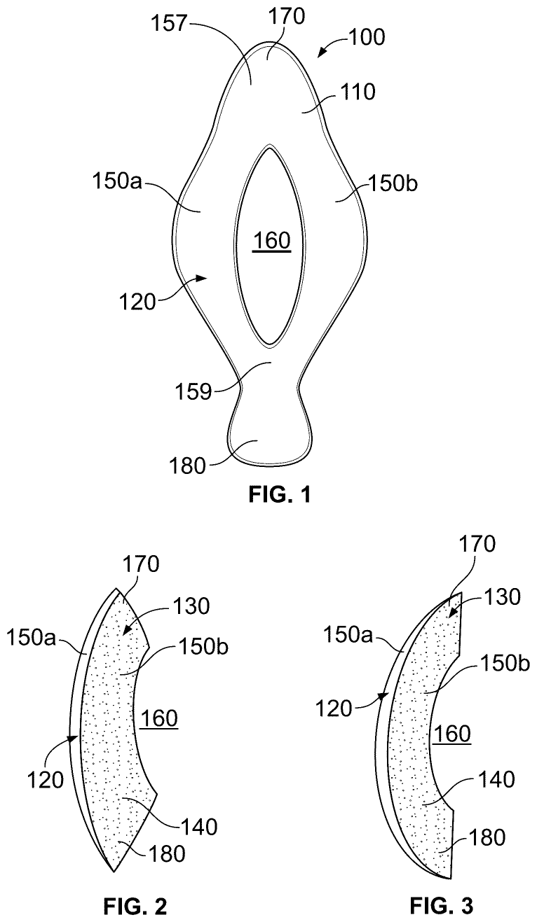

Referring now to FIGS. 1 and 2, an exemplary labial retention device 100 is shown for illustrative purposes. As will be appreciated from FIGS. 1 and 2, the labial retention device 100 may be formed as a unitary body 110 having a front (clinician-facing) side surface 120 and a rear (patient-facing) side surface 130. The body may be curved in 1, 2 or 3 orthogonal planes, e.g., to provide a saddle-like overall shape.

The body may be constructed of a pliable material such as medical-grade silicone, however, the body must be rigid enough to retain the labia in retracted positions sufficient for visualization purposes, as described in greater detail below.

The rear side surface 130 of the labial retention device 100 is provided with a mild/low-tack medical-grade adhesive 140 suitable for adhering to skin, and not so tacky as to be likely to cause skin damage or undue discomfort during removal of the device from the skin. The adhesive 140 may be provided as a coating or otherwise over an entirety of the rear side surface 130, or only on discrete portions. Optionally, a release sheet may be provided to protect the adhesive from contact until after removal of the release sheet.

The body 110 defines a pair of laterally opposed wings 150a, 150b shaped to define a central opening 160 therebetween that is shaped and dimensioned to allow for visualization of both the urethra U and vagina V of typical human female anatomy when the device 100 is in use in contact with such typical human female (e.g., adult human female) anatomy. For example, the central opening may be approximately 2.0 to 2.5 inches tall and approximately 0.5 to 1.0 inches wide. In this exemplary embodiment, the wings 150a, 150b of the body 110 cooperate to form a closed loop that defines the central opening 160 internally to the body 110. The wings 150a, 150b meet at first and second junctions 157, 159, and define therebetween the central opening sized 160, as best shown in FIG. 1.

The body 110 further defines an anterior tab 170 sized and shaped to overly at least a portion of the prepuce and/or clitoris C of typical human (e.g., adult) female anatomy when the device is in use in contact with such typical human female anatomy. The anterior tab 170 is joined to the first junction 157 of the wings 150a, 150b and extends in a first direction, away from the central opening 160, as best shown in FIG. 1. For example, the anterior tab 170 may extend approximately 1 to 2 inches from a terminal anterior point of the central opening 160. A hood may be provided on the anterior tab 170 to assist in retracting the skin above the clitoris more effectively.

In this exemplary embodiment, the body 110 further defines a posterior tab 180 sized and shaped to overlie at least a portion of the perineum and/or anus of typical human female anatomy when the device is in use in contact with such typical human female anatomy. The posterior tab 180 is joined to the second junction 159 of the wings 150a, 150b and extends in a second direction, opposite the first direction, away from the central opening 160, as shown in FIG. 1.

For example, the posterior tab 180 may extend approximately 0.3 to 0.8 inches from a terminal posterior point of the central opening 160.

FIG. 3 is a right side view of an alternative labial retention device 100 in accordance with an alternative embodiment of the present invention. This alternative labial retention device 100 includes the same elements described above, but has a different overall three-dimensional shape by way of example for illustrative purposes.

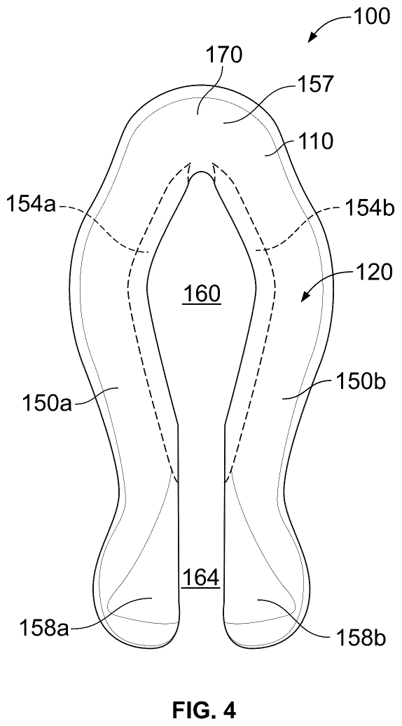

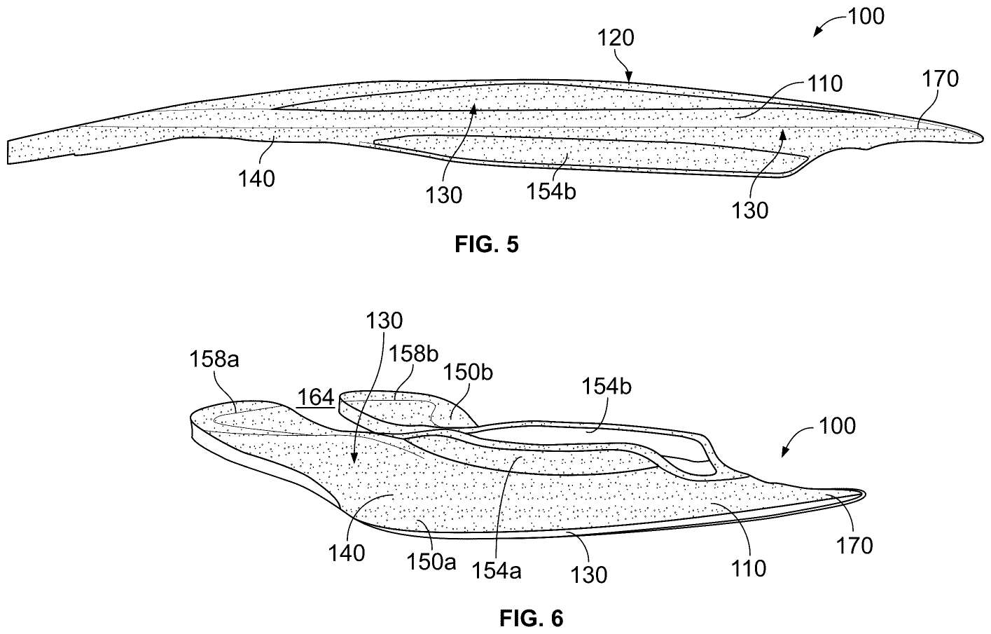

FIGS. 4-6 show another alternative labial retention device 100 in accordance with another alternative embodiment of the present invention. This embodiment is similar to those of FIGS. 1-3. However, in this exemplary embodiment, the wings 150a, 150b of the body 110 meet at a first junction 157 but do not form a closed loop, and rather cooperate to define a central opening 160 that is not internal to the body 110, but rather is open to a space external to the body 110 via a gap 164. Accordingly, the body 110 is generally U-shaped, as may be best appreciated from FIG. 4. Accordingly, in this embodiment, the device 100 may not include a posterior tab. The gap 164 facilitates removal of the device 100 from the body, after catheter insertion, without interference with the catheter, as discussed in greater detail below.

Further, in this exemplary embodiment, the body 110/pair of wings 150a, 150b further defines a pair of sidewalls 154a, 154b. The sidewalls 154a, 154b extend generally transversely to the front side surface 120, along/away from the rear side surface 130, as may be best appreciated from FIGS. 4-6. In use, the sidewalls 154a, 154b act somewhat like a dam to interfere with and retain the labia in displaced positions to aid in visualization during catheter placement, as discussed in greater detail below. The adhesive 140 on the rear side of the body 110 may be provided on the bottom/side surfaces of the sidewalls 154a, 154b.

Further, in this exemplary embodiment, each of the wings 150a, 150b of the body 110 of the device 100 defines an elongated tab 158a, 158b. The elongated tabs 158a, 158b extend generally in a place of the body/wings, but are elongated to be likely to extend beyond the labia when the device is positioned on the body. In use, the elongated tabs 158a, 158b may be manually grasped and pulled to facilitate removal of the device 100 from the body, as discussed in greater detail below.

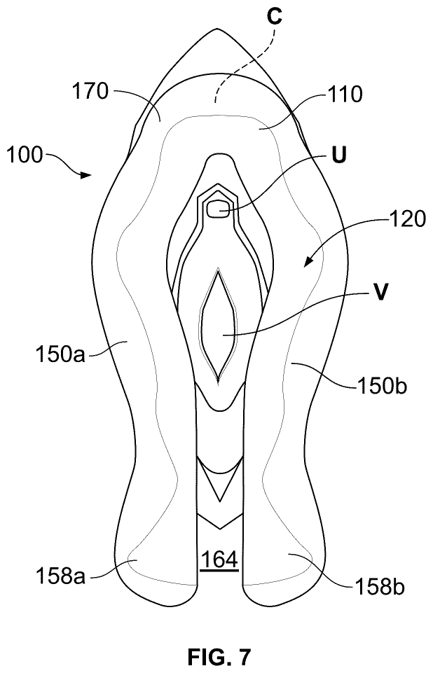

FIG. 7 is a front view of the labial retention device 100 of FIG. 4, shown in use. The device 100 of FIGS. 1-3 may be used in a similar manner.

The labial retention device 100 for female urinary catheterization may be used as a single-use/new use item, such that it may be used for a single procedure for a single patient, and then be discarded.

In use, consistent with recognized appropriate catheterization procedures, a nurse/clinician should first wash/sterilize the clinician's hands. Then, for effective use of the labial retention device, the genitals/labia, and surrounding tissue should first be cleaned with soap and water (or a suitable wipe/towelette), and be air-dried or patted dry.

Next, a skin-preserving preparation (such as that sold as Cavilon Barrier Cream) may be applied on intact skin, for the purpose of protecting the skin against friction or shearing when the labial retention device is removed. For women with low pain/discomfort tolerance, she may take an oral over-the-counter anesthetic such as Tylenol, or obtain a prescription for a topical anesthetic, such as lidocaine jelly, which may be applied to the labia and/or surrounding tissues prior to device application to mitigate any discomfort resulting from use of the device.

The clinician should then again wash/sanitize the clinician's hands, then remove the labial retention device 100 from any kit of packaging. A release sheet covering the adhesive 140 may be removed, if provided.

Next, using a clean technique, the labial retention device 100 is applied to the person's urogenital region. More particularly, the labial retention device 100 may be placed against or adjacent skin of the urogenital cleft, with the adhesive-bearing rear side 130 facing the person's skin, with the anterior tab 170 adjacent to the clitoris C and the posterior tab 180 (if any) adjacent to the perineum, as will be best appreciated from FIG. 7.

The hood of the clitoris/prepuce C may then be retracted and adhered to the anterior tab 170 to assist in exposing the urethra U for optimal visualization.

Further, each of the bilateral labia (labia minor and/or labia majora) may then be stretched or pulled up and to the bilateral sides of the device 100, around the sidewalls 154a, 154b, if provided, and be placed/pressed into contact with the adhesive 140 on the rear side surface 130 of the wings 150a, 150b. This causes the labia to be adhered to the labial retention device 100, and to thereby be not only retracted but also retained in open/spread/displaced positions by the wings 150a, 150b and/or sidewalls 154a, 154b, of the body 110 of the labial retention device 100. In this manner, the device 100 provides a clear view of the vaginal V and urethral U openings/regions (through the central opening 160 of the labial retention device 100) in a hands-free manner, without the need for continued use of the clinician's hands after the labial retention device 100 has been properly applied to the body, as will be best appreciated from FIG. 7.

Accordingly, the labial retention device 100 effectively performs the work of the clinician's non-dominant hand (to spread the labia in conventional procedures), freeing both of the clinician's hands for use in employing a sterile technique to insert the catheter in the proper (urethral U) opening to minimize the risk of contamination.

A Foley (or other) urinary catheter may then be placed by insertion into the urethra U consistent with conventional catheter insertion procedures, but with the use of both of the clinician's hands, while the labial retention device 100 retains the labia in displaced positions for visualization purposes.

At an appropriate point after completion of the catheterization process, the catheter may be removed from the urethra, and then the device may be removed from the skin by peeling the device away from the bodily tissues to release the tissues from the adhesive. Alternatively, before removal of the catheter, the devices 100 of FIGS. 1-3 may be removed by threading an end of the catheter/catheter assembly though the central opening 160. Alternatively, before removal of the catheter, the device of FIGS. 4-7, may be removed by movement of the device 100 in a direction transverse to an axis of elongation of the catheter, such that the catheter passes through the gap 164 defined between ends of the wings 150a, 150b, as will be best appreciated from FIGS. 4, 6 and 7. In this embodiment, grasping a free end of the elongated tabs 158a/158b may facilitate peeling of the device 100 away from the skin to release the device 100 from the bodily tissues.

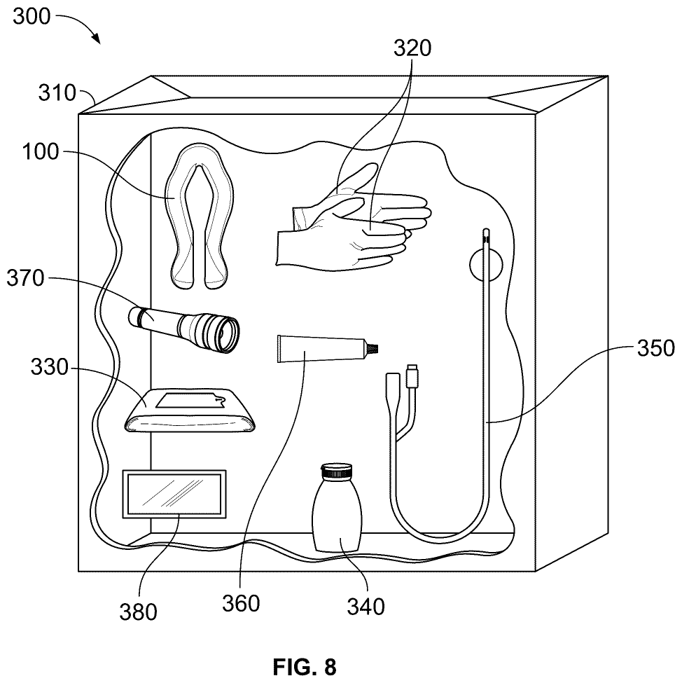

Consistent with the present invention, also provided is an exemplary catheterization kit 300 intended for clinician use, including at least one labial retention device 100, and additional items that are typically used/needed by a clinician in a urinary catheterization procedure. For example, as shown in FIG. 8, one exemplary kit 300 includes, in a common package 310, at least one labial retention device 100, at least one pair of medical-use (sterile or non-sterile) gloves 320, at least one moistened cleansing towelette 330, and a package of skin-preserving preparation 340 (e.g., on or with an applicator pad). In certain embodiments, a catheter 350 (e.g., Foley catheter) and a package of skin lubricant 360 may also be included.

Consistent with the present invention, also provided is a catheterization kit suitable for use with patients for self-catheterization, including at least one labial retention device 100, and additional items that are typically used/needed in a urinary self-catheterization procedure. For example, as shown in FIG. 8, one exemplary kit 300 includes, in a common package 310, at least one labial retention device 100, at least one pair of medical-use gloves 320, at least one moistened cleansing towelette 330, and at least one package of skin-preserving preparation 340. In certain embodiments, a catheter 350, package of skin lubricant 360, an illumination source 370 (e.g., light/lamp/flashlight), and a mirror 380 are also included.

In certain embodiments, the labial retention devices are configured to have a self-nesting shape, such as a three-dimensional saddle shape, that allows a plurality of them to be stacked in a compact nested configuration, for shipping and/or storage purposes.

While the primary purpose of the labial retention device is for use in a urinary catheterization process, it should be noted that the labial retention device may be used in other medical contexts for other medical procedures, and further may also be used in non-medical contexts for other purposes, such as female sexual health purposes.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A labial retention device comprising:
a pair of wings joined at least at a first junction and defining therebetween a central opening sized and shaped to permit visualization of the urethra and vaginal openings of typical human female anatomy through the central opening when the device is in contact with a genital region of female anatomy; and
an anterior tab joined to said pair of wings at said first junction and extending in a first direction away from said central opening.

2. The labial retention device of claim 1, wherein said pair of wings is further joined at a second junction opposite said first junction, said pair of forming a closed loop around said central opening.

3. The labial retention device of claim 2, further comprising:
a posterior tab joined to said pair of wings at said second junction, said posterior tab extending in a second direction, opposite said first direction, away from said central opening.

4. The labial retention device of claim 1, wherein said pair of wings and said anterior tab are formed as a unitary body.

5. The labial retention device of claim 4, wherein said body is curved relative to at least one plane.

6. The labial retention device of claim 4, wherein said body is curved relative to at least two orthogonal planes.

7. The labial retention device of claim 4, wherein said body is constructed of a medical-grade silicone material.

8. The labial retention device of claim 4, wherein said body defines a front surface and a rear surface, and wherein a skin-adhering adhesive supported on said rear surface.

9. The labial retention device of claim 1, wherein each of said pair of wings has a front surface and a rear surface and defines a respective sidewall extending generally transversely to the front surface and away from the rear surface.

10. The labial retention device of claim 1, wherein each of said pair of wings defines an elongated tab elongated to extend beyond the typical human female labia when the device is in contact with a genital region of female anatomy.

11. A labial retention device comprising:
a body defining a pair of wings joined at least at a first junction and defining therebetween a central opening sized and shaped to permit visualization of the urethra and vaginal openings of typical human female anatomy through the central opening when the device is in contact with a genital region of female anatomy, said body having a front surface and a rear surface; and
adhesive supported on said rear surface of said body.

12. The labial retention device of claim 11, further comprising:
an anterior tab joined to said pair of wings at said first junction and extending in a first direction away from said central opening; and
a posterior tab joined to said pair of wings at said second junction and extending in a second direction, opposite said first direction, away from said central opening.

13. The labial retention device of claim 11, wherein said pair of wings is further joined at a second junction opposite said first junction, said pair of forming a closed loop around said central opening.

14. The labial retention device of claim 11, wherein said body is curved relative to at least one plane.

15. The labial retention device of claim 11, wherein said body is constructed of a pliable material.

16. The labial retention device of claim 11, wherein each of said pair of wings defines a respective sidewall extending generally transversely to the front surface and away from the rear surface.

17. The labial retention device of claim 11, wherein each of said pair of wings defines an elongated tab elongated to extend beyond the typical human female labia when the device is in contact with a genital region of female anatomy.

18. A catheterization kit comprising:
a labial retention device comprising:
a body defining a pair of wings joined at least at a first junction and defining therebetween a central opening sized and shaped to permit visualization of the urethra and vaginal openings of typical human female anatomy through the central opening when the device is in contact with a genital region of female anatomy, said body having a front surface and a rear surface; and
adhesive supported on said rear surface of said body; and
a pair of gloves;
a moistened cleansing towelette; and
a skin-preserving preparation.

19. The catheterization kit of claim 18, further comprising:
at least one of a urinary catheter and a skin lubricant.

20. The catheterization kit of claim 18, further comprising:
a package defining an internal volume, each of said labial retention device, said pair of gloves, said moistened cleaning towelette, and said skin preparation material being disposed in said internal volume of said package.

21. The catheterization kit of claim 18, further comprising:
at least one of a mirror and an illumination source.
